(19) United States
(12) Patent Application Publication
(10) Pub. No.: US 2026/0183185 A1
(43) Pub. Date: Jul. 2, 2026

(54) SHOE FROM WHICH PULSED ELECTROMAGNETIC FIELD IS GENERATED

(71) Applicant: Huelight Co., Ltd., Gwangmyeong-si (KR)

(72) Inventor: Sung Youn LEE, Gwangmyeong-si (KR)

(21) Appl. No.: 19/253,983

(22) Filed: Jun. 30, 2025

(30) Foreign Application Priority Data
Dec. 31, 2024 (KR) 10-2024-0201881

(51) Int. Cl. A61H 23/02; A43B 3/44; G16H 40/67

(52) U.S. Cl. CPC A61H 23/0218; A43B 3/44; G16H 40/67; A61H 2201/1642; A61H 2201/165; A61H 2201/5007; A61H 2205/12; A61H 2230/625

(57) ABSTRACT

According to one embodiment of the present invention, a shoe using a pulsed electromagnetic field includes a shoe body, a sole provided below the shoe body, a battery module provided on the sole and configured to supply DC power, an electromagnetic field vibration device and a sensing module provided on the sole and connected to the battery module through a cable. While a user wearing the shoe walks, the sensing module calculates a walking pattern based on sensing data. The electromagnetic field vibration device includes a plurality of coils and a control unit disposed to overlap a narrowest portion of a middle portion of the sole. The coils are disposed at a front end portion and a rear end portion of the sole and connected to the control unit through a cable.

### CROSS-REFERENCE TO RELATED APPLICATION

This application claims priority to and the benefit of Korean Patent Application No. 10-2024-0201881, filed on Dec. 31, 2024, the disclosure of which is incorporated herein by reference in its entirety.

### BACKGROUND

### 1. Field of the Invention

The present invention relates to a shoe from which a pulsed electromagnetic field is generated, and more specifically, to a shoe from which a pulsed electromagnetic field is generated, and the output of the pulsed electromagnetic field is controlled to be customized according to the walking characteristics of a pedestrian.

### 2. Discussion of Related Art

While continuously standing or walking for long periods of time in daily life, modern people often feel tired in their feet or have difficulty with blood circulation. In particular, the impediment to blood circulation may cause various health problems, and various methods are being tried to solve these problems. Among the various methods, there is a technology for improving blood circulation by using electrical stimulation or vibrations on the feet.

Since existing blood circulation promotion devices or vibration massage devices are generally separate mechanical devices that are mainly placed on the floor and used or should be directly attached to the feet and used, the blood circulation promotion devices or vibration massage devices lack portability and restrict the user's range of activity. In addition, there is a problem in that these devices lack availability in daily life due to dependence on external power or a heavy and cumbersome structure.

In the present invention, in order to reduce such problems, a shoe from which a pulsed electromagnetic field is generated, and an electromagnetic field is generated in the form of a short pulse (wave) according to the walking characteristics of a user to allow the user to relieve foot fatigue in real time while walking, will be described.

### Related Art Documents

Korean Laid-open Patent No. 10-2016-0095479

### SUMMARY OF THE INVENTION

The present invention is directed to providing a shoe from which a pulsed electromagnetic field is generated, and more specifically, to providing a shoe from which a pulsed electromagnetic field is generated, and the output of a pulsed electromagnetic field is controlled to be customized according to the walking characteristics of a pedestrian.

According to an aspect of the present invention, there is provided a shoe using a pulsed electromagnetic field technology for generating vibrations using a pulsed electromagnetic field, which includes a shoe body, a sole provided below the shoe body, a battery module provided on the sole and configured to supply DC power, an electromagnetic field vibration device provided on the sole and connected to the battery module through a cable, and a sensing module provided on the sole and connected to the battery module through a cable, wherein, while a user wearing the shoe walks, the sensing module calculates a walking pattern of a pedestrian based on sensing data transmitted from the shoe, the electromagnetic field vibration device includes a plurality of coils and a control unit configured to control the plurality of coils, the control unit of the electromagnetic field vibration device is disposed to overlap a narrowest portion of a middle portion of the sole, the plurality of coils of the electromagnetic field vibration device are disposed to be distributed at a front end portion of the sole and a rear end portion of the sole and are connected to the control unit of the electromagnetic field vibration device through a cable, and the control unit of the electromagnetic field vibration device generates a pulsed electromagnetic field in different manners through the plurality of coils according to the calculated walking pattern.

The shoe may further include a switch module connected to the battery module through a cable to control the electromagnetic field vibration device to be turned on or off, wherein the control unit is connected to a pedestrian terminal used by the pedestrian through a network by using a provided communication module and transmits operation information indicating an operation state of the plurality of coils to the pedestrian terminal in real time, the shoe body includes a fixture for attachment and separation of the switch module, when a time for which the user wearing the shoe walks satisfies a set time, the sensing module calculates the walking pattern of the pedestrian based on the sensing data during the time, the walking pattern includes information about a foot landing method, a pressure distribution during walking, a walking rhythm, a walking speed, and a stride, and the number of the coils disposed at the front end portion of the sole is greater than the number of the coils disposed at the rear end portion of the sole.

The foot landing method of the walking pattern may be classified into a front end landing method, a middle landing method, and a rear end landing method, the pressure distribution during walking of the walking pattern may be classified into a uniform distribution, an inner dense distribution, and an outer dense distribution, the walking rhythm of the walking pattern may be classified into a regular walking rhythm which has a constant walking speed and stride and an irregular walking rhythm which has an irregular walking speed and stride, the walking speed of the walking pattern may be classified into a high speed, an average speed, and a low speed, and the stride of the walking pattern may be classified into a long stride, an average stride, and a short stride.

As the foot landing method of the pedestrian is closer to the front end landing method, the control unit of the electromagnetic field vibration device may generate a first control signal for causing a higher current to flow in the coils disposed at the front end portion of the sole as compared to other coils, as the pressure distribution during walking of the pedestrian is closer to the inner dense distribution, the control unit of the electromagnetic field vibration device may generates a second control signal for performing control such that a higher current flows in the coils disposed at an inner side of the sole as compared to other coils, as the walking speed during walking of the pedestrian is closer to the high walking speed, the control unit of the electromagnetic field vibration device may generate a third control signal for performing control such that a pulse period of a current flowing in the coil provided on the sole becomes shorter, as the stride during walking of the pedestrian becomes longer, the control unit of the electromagnetic field vibration device may generate a fourth control signal for performing control such that intensity of a current flowing in the coil provided on the sole is increased, and the plurality of coils of the electromagnetic field vibration device may be operated by overlapping and reflecting the first to fourth control signals.

When the walking rhythm during walking of the pedestrian is the regular walking rhythm, the control unit of the electromagnetic field vibration device may normally generate the third control signal and the fourth control signal according to a walking speed and a stride during walking of the pedestrian, when the walking rhythm during walking of the pedestrian is the irregular walking rhythm, the control unit of the electromagnetic field vibration device may block the third control signal and the fourth control signal and may calculate each of a speed range and a stride range based on a maximum value and a minimum value of the walking speed of the pedestrian and a maximum value and a minimum value of the stride of the pedestrian, when the walking rhythm during walking of the pedestrian is the irregular walking rhythm, the control unit of the electromagnetic field vibration device may calculate a current pulse period corresponding to a maximum value of a calculated speed range and a current pulse period corresponding to a minimum value of the calculated speed range based on a process of generating the third control signal and may generate a first alternating signal for causing currents with a pair of current pulse periods to be alternately applied to the coil provided on the sole at a set interval, the control unit of the electromagnetic field vibration device may calculate an intensity value of a current corresponding to a maximum value of a calculated stride range and an intensity value of a current corresponding to a minimum value of the calculated stride range based on a process of generating the fourth control signal and may generate a second alternating signal for causing currents with a pair of intensity values to be alternately applied to the coil provided on the sole at a set interval, the plurality of coils of the electromagnetic field vibration device may be operated by overlapping and reflecting the first control signal, the second control signal, the first alternating signal, and the second alternating signal, and the set interval may be an interval of 1 second or less.

The shoe may further include a plurality of reinforcing bands which include an elastic material, are connected to the control unit, extend from the sole, and include coils at end portions thereof, wherein a plurality of connectors connected to the control unit through a cable and a plurality of dummy connectors which have the same shape as the connectors and are electrically blocked are provided at portions of an outer surface of the shoe body adjacent to the sole, the connector and the dummy connector are provided adjacent to each other to form a pair of connector groups on each of an outer surface and an inner surface of each of a front side portion and a rear side portion of the shoe body, the connector and the dummy connector are provided adjacent to each other to form a pair of connector groups on a rear surface of the shoe body, a band hole through which the reinforcing band passes is formed in a portion of the sole adjacent to each of a plurality of connector groups, the pedestrian connects each reinforcing band, which passes through the band hole to protrude and extend to the outside, to any one of the connector and the dummy connector of an adjacent connector group, when the pedestrian connects the end portion of the reinforcing band to an adjacent connector, the coil of the end portion of the reinforcing band is electrically connected to the control unit so that a pulsed electromagnetic field is formed around the coil, and when the pedestrian connects the end portion of the reinforcing band to an adjacent dummy connector, the coil of the end portion of the reinforcing band is electrically blocked from the control unit.

As the foot landing method of the pedestrian is closer to the front end landing method, the control unit of the electromagnetic field vibration device may transmit a first recommendation signal to the pedestrian terminal, wherein the first recommendation signal is for recommending that an adjacent reinforcing band is connected to the connector included in the connector group provided at the front side portion of the shoe body, and the remaining reinforcing band is connected to the dummy connector in an adjacent connector group, as the foot landing method of the pedestrian is closer to the rear end landing method, the control unit of the electromagnetic field vibration device transmits the first recommendation signal to the pedestrian terminal, wherein the first recommendation signal is for recommending that the adjacent reinforcing band is connected to the connector included in the connector group provided at each of the rear side portion and the rear surface of the shoe body, and the remaining reinforcing band is connected to the dummy connector in the adjacent connector group, as the pressure distribution during walking of the pedestrian is closer to the inner dense distribution, the control unit of the electromagnetic field vibration device transmits a second recommendation signal to the pedestrian terminal, wherein the second recommendation signal is for recommending that the adjacent reinforcing band is connected to the connector included in the connector group provided on the inner surface of each of the front side portion and the rear side portion of the shoe body, and the remaining reinforcing band is connected to the dummy connector in the adjacent connector group, as the pressure distribution during walking of the pedestrian is closer to the outer dense distribution, the control unit of the electromagnetic field vibration device transmits the second recommendation signal to the pedestrian terminal, wherein the second recommendation signal is for recommending that the adjacent reinforcing band is connected to the connector included in the connector group provided on the outer surface of each of the front side portion and the rear side portion of the shoe body, and the remaining reinforcing band is connected to the dummy connector in the adjacent connector group, the connector has an upper surface that protrudes downward from the outer surface of the shoe body to form a predetermined acute angle with the outer surface of the shoe body and has a hole into which the end portion of the reinforcing band is inserted, and a lower surface configured to connect an end portion of the upper surface and the outer surface of the shoe body, when the end portion of the reinforcing band is inserted into the hole formed in the upper surface of the connector, the coil provided at the end portion of the reinforcing band is fastened to the connector and electrically connected to the control unit, and when the end portion of the reinforcing band is fastened to the connector, a direction of a virtual central axis of an electromagnetic field generated at the coil provided at the end portion of the reinforcing band is a direction perpendicular to the upper surface of the connector to be directed to an inner bottom surface of the shoe body.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a perspective view of a shoe including an electromagnetic field vibration device according to one embodiment of the present invention;

FIG. 2 is a view for describing a connection relationship between components provided in a sole of the shoe including the electromagnetic field vibration device according to one embodiment of the present invention;

FIG. 3 is a block diagram illustrating a state in which components related to the shoe including the electromagnetic field vibration device according to one embodiment of the present invention are connected through a network;

FIG. 4 is a side view of the shoe including the electromagnetic field vibration device according to one embodiment of the present invention;

FIG. 5 is a rear view of the shoe including the electromagnetic field vibration device according to one embodiment of the present invention;

FIG. 6A is a side view illustrating the shoe including the electromagnetic field vibration device according to one embodiment of the present invention in a state in which a reinforcing band is not coupled to a connector;

FIG. 6B is a rear view illustrating the shoe including the electromagnetic field vibration device according to one embodiment of the present invention in the state in which the reinforcing band is not coupled to the connector; and

FIG. 7 is an enlarged view illustrating a state in which the reinforcing band (SB) is connected to the connector (CN) according to one embodiment of the present invention.

### DETAILED DESCRIPTION OF EXEMPLARY EMBODIMENTS

Hereinafter, embodiments of the present invention will be described in detail with reference to the accompanying drawings so that those skilled in the art can carry out the present invention. However, the present invention may be implemented in various forms and is not limited to the embodiments described herein. In order to clearly explain the present invention in the drawings, parts not related to the description are omitted, and similar parts are given similar reference numerals throughout the specification.

Throughout the specification, it will be understood that when a first element is referred to as being “coupled” or “connected” to a second element, the first element can be directly coupled or electrically connected to the second element or intervening elements may be present therebetween. In addition, throughout the specification, unless explicitly described to the contrary, the word “include” and variations such as “comprise” or “comprising” will be understood to imply the inclusion of stated elements but not the exclusion of any other elements. The present invention will be described in detail below with reference to the accompanying drawings.

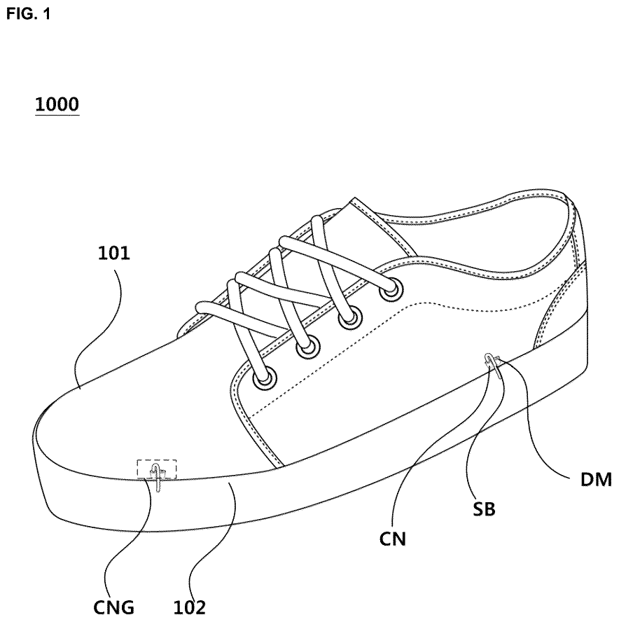

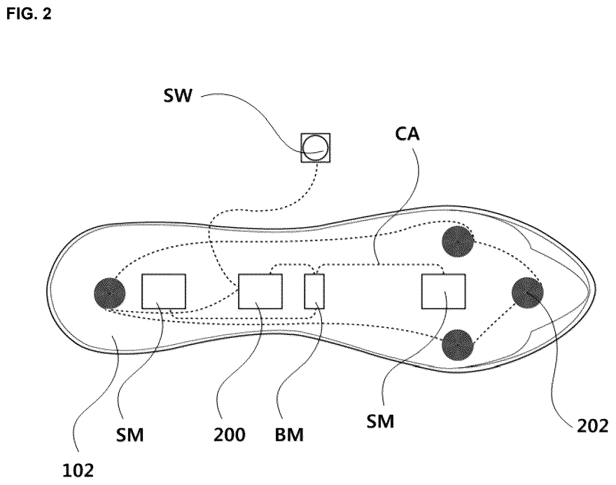

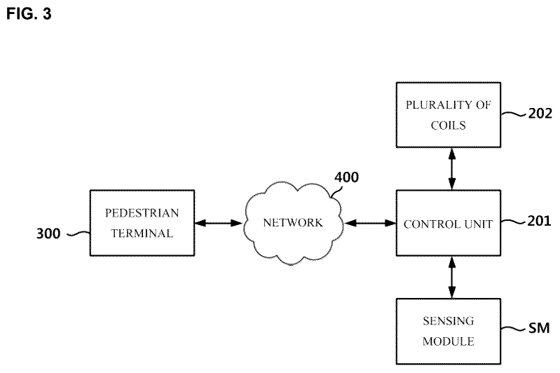

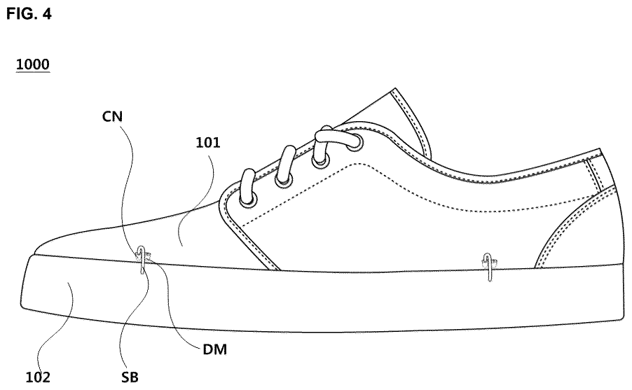

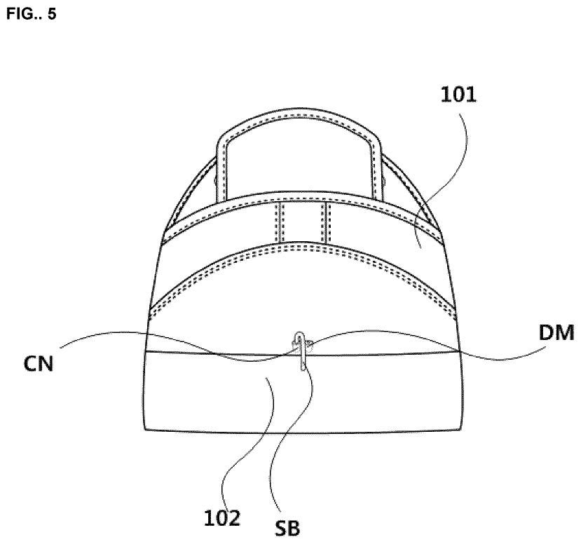

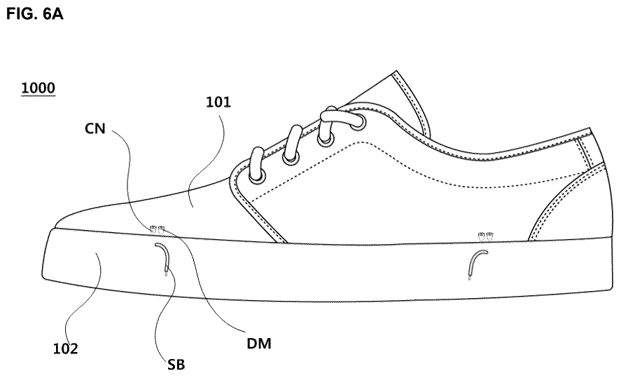

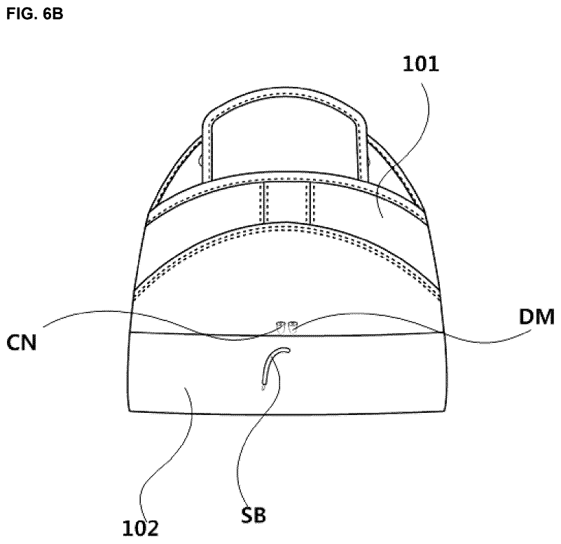

FIG. 1 is a perspective view of a shoe 1000 including an electromagnetic field vibration device according to one embodiment of the present invention. FIG. 2 is a view for describing a connection relationship between components provided in a sole 102 of the shoe 1000 including the electromagnetic field vibration device according to one embodiment of the present invention. FIG. 3 is a block diagram illustrating a state in which components related to the shoe 1000 including the electromagnetic field vibration device according to one embodiment of the present invention are connected through a network 400. FIG. 4 is a side view of the shoe 1000 including the electromagnetic field vibration device according to one embodiment of the present invention. FIG. 5 is a rear view of the shoe 1000 including the electromagnetic field vibration device according to one embodiment of the present invention. FIG. 6A is a side view illustrating the shoe 1000 including the electromagnetic field vibration device according to one embodiment of the present invention in a state in which a reinforcing band SB is not coupled to a connector CN. FIG. 6B is a rear view illustrating the shoe 1000 including the electromagnetic field vibration device according to one embodiment of the present invention in the state in which the reinforcing band SB is not coupled to the connector CN.

Referring to FIGS. 1 to 5, the shoe 1000 according to one embodiment of the present invention may include a shoe body 101, the sole 102, a battery module BM, an electromagnetic field vibration device 200, a sensing module SM, and a switch module SW.

The shoe body 101 may be a body of a shoe that a person typically wears.

The sole 102 may be provided below the shoe body 101.

The battery module BM may be provided on the sole 102 and may supply DC power.

The electromagnetic field vibration device 200 may be provided on the sole 102 and may be electrically connected to the battery module BM through a cable CA.

The sensing module SM may be provided on the sole 102 and may be electrically connected to the battery module BM through the cable CA.

The switch module SW may be connected to the electromagnetic field vibration device 200 through the cable CA and may control the tuning-on/off of the electromagnetic field vibration device 200. The switch module SW may be provided on a side surface of the sole 102 or in the shoe body 101, and in this case, a fixture (not shown) for attachment and separation of the switch module may be provided on the side surface of the sole 102 or in the shoe body 101. A pedestrian may control the electromagnetic field vibration device 200 to be turned on or off by switching the switch module.

In FIG. 2, for convenience of description, the battery module BM, the electromagnetic field vibration device 200, and the sensing module SM are illustrated as being attached to an outer surface of the sole 102, but the battery module BM, the electromagnetic field vibration device 200, the sensing module SM, and the cable CA electrically connecting the battery module BM, the electromagnetic field vibration device 200, and the sensing module SM may be provided to be embedded in the sole 102 to reduce an impact from the outside. However, the present invention is not limited thereto, and the battery module BM, the electromagnetic field vibration device 200, the sensing module SM, and the cable CA may be provided to be attached to a bottom surface of the sole 102.

The electromagnetic field vibration device 200 and the sensing module SM may be driven by DC power supplied by the battery module BM.

The electromagnetic field vibration device 200 may include a plurality of coils 202 and a control unit 201 that controls the plurality of coils 202.

The control unit 201 may be connected to a pedestrian terminal 300 used by a pedestrian through the network 400 by using a provided communication module and may transmit operation information indicating an operation state of the plurality of coils 202 to the pedestrian terminal 300 in real time. A pedestrian may check the operation state of the plurality of coils 202 through the pedestrian terminal 300 to check his or her walking style in real time.

The plurality of coils 202 of the electromagnetic field vibration device 200 may be distributed and disposed at each of a front end portion of the sole 102 and a rear end portion of the sole 102, and the coils 202 may be connected to the control unit 201 of the electromagnetic field vibration device 200 through the cable CA. As shown in FIG. 2, the number of coils 202 disposed at the front end portion of the sole 102 may be greater than the number of coils 202 disposed at the rear end portion of the sole 102. This is because it is contemplated in the sole 102 that an area of a rear end portion of each of a sole of a foot of a pedestrian and a bottom surface of the sole 102 is generally greater than an area of a front end portion thereof.

The control unit 201 of the electromagnetic field vibration device 200 may be deposed to overlap the narrowest portion of a middle portion of the sole 102 such that a length of the cable CA connected to the plurality of coils 202 is not biased to one portion.

The sensing module SM may calculate a walking pattern of a pedestrian who wears shoes of the present invention. While a pedestrian wearing the shoe walks, the sensing module SM may calculate a walking pattern of the pedestrian based on sensing data transmitted from the shoes.

When a time for which a user wearing the shoe walks satisfies a set time, the sensing module SM may calculate a walking pattern of a pedestrian based on sensing data during the time. As an example of the present invention, the sensing module SM may include a pressure sensor for measuring a pressure distribution and a landing method of a sole of a foot, an inertial measurement unit (IMU) for measuring a walking rhythm, a speed, an interval, or the like, and a walking pattern may include information about a foot landing method, a pressure distribution during walking, a walking rhythm, a walking speed, or a stride. A walking rhythm of a walking pattern may be classified into a regular walking rhythm which has a constant walking speed and stride and an irregular walking rhythm which has an irregular walking speed and stride, a walking speed of a walking pattern may be classified into a high speed, an average speed, and a low speed, and a stride of a walking pattern may be classified into a long stride, an average stride, and a short stride.

The control unit 201 of the electromagnetic field vibration device 200 may drive the plurality of coils 202 in different manners according to the calculated walking pattern. Hereinafter, how the plurality of coils 202 are driven according to the calculated walking pattern will be described in detail.

As a foot landing method of a pedestrian is closer to a front end landing method, the control unit 201 of the electromagnetic field vibration device 200 may generate a first control signal for causing a higher current to flow in the coils 202 disposed at the front end portion of the sole 102 as compared to other coils 202. When a foot landing method of a pedestrian is close to a front end landing method, since a front portion of a sole of a foot may come into a lot of contact with a ground surface and thus may be more fatigued than other portions, the control unit 201 may cause a higher current to flow in the coils 202 provided at the front end portion of the sole 102, and thus vibrations due to a stronger pulse electromagnetic field may be generated in the coils 202 disposed at the front end portion of the sole 102 as compared to other portions, thereby effectively reducing fatigue of the front portion of the sole of the pedestrian.

On the other hand, as a foot landing method of a pedestrian is closer to a rear end landing method, the control unit 201 of the electromagnetic field vibration device 200 may generate the first control signal for causing a higher current to flow in the coils 202 disposed at the rear end portion of the sole 102.

As a pressure distribution during walking of a pedestrian is closer to an inner dense distribution, the control unit 201 of the electromagnetic field vibration device 200 may generate a second control signal for performing control such that a higher current flows in the coils 202 disposed at an inner portion of the sole 102 as compared to other coils 202. When a pressure distribution during walking of a pedestrian is close to an inner dense distribution, since an inner portion of a sole of a foot may come into a lot of contact with a ground surface and thus may be more fatigued than other portions, the control unit 201 may cause a higher current to flow in the coils 202 provided at the inner portion of the sole 102, and thus vibrations due to a stronger pulse electromagnetic field may be generated in the coils 202 disposed at the inner portion of the sole 102 as compared to other portions, thereby effectively reducing fatigue of the inner portion of the sole of the foot of the pedestrian.

On the other hand, as a pressure distribution during walking of a pedestrian is closer to an outer dense distribution, the control unit 201 of the electromagnetic field vibration device 200 may generate the second control signal for causing a higher current to flow in the coils 202 provided at an outer portion of the sole 102.

As a walking speed during walking of a pedestrian is closer to a high walking speed, the control unit 201 of the electromagnetic field vibration device 200 may generate a third control signal for performing control such that a pulse period of a current flowing in the coil 202 provided on the sole 102 becomes shorter. When a walking speed of a pedestrian is high, the number of times a sole of a foot touches a ground surface in the same amount of time may be considered to be greater than an average number. In response, to increase the number of times of generation of a pulsed electromagnetic field at the coil 202, the control unit 201 of the electromagnetic field vibration device 200 may shorten a pulse period of a current flowing in the coil 202, thereby effectively reducing fatigue of a foot of a pedestrian with a high walking speed.

On the other hand, as a walking speed during walking of a pedestrian is closer to a low walking speed, the control unit 201 of the electromagnetic field vibration device 200 may generate the third control signal for performing control such that a pulse period of a current flowing in the coil 202 provided on the sole 102 becomes longer.

As a stride during walking of a pedestrian becomes longer, the control unit 201 of the electromagnetic field vibration device 200 may generate a fourth control signal for performing control such that the intensity of a current flowing in the coil 202 provided on the sole 102 is increased. When a stride of a pedestrian is long, since a force with which a sole of a foot of the pedestrian presses a ground surface may be considered to be strong, in response, the control unit 201 of the electromagnetic field vibration device 200 increases the intensity of a current flowing in the coil 202 such that a stronger pulsed electromagnetic field is generated at the coil 202, thereby effectively reducing fatigue of a foot accumulated as the pedestrian walks in strong contact with the ground surface.

On the other hand, as a stride during walking of a pedestrian becomes shorter, the control unit 201 of the electromagnetic field vibration device 200 may generate the fourth control signal for performing control such that the intensity of a current flowing in the coil 202 provided on the sole 102 is decreased.

The plurality of coils 202 of the electromagnetic field vibration device 200 may be operated by overlapping and reflecting the first to fourth control signals. Since the first to fourth control signals are driven independently, the plurality of coils 202 may be driven by reflecting all the first to fourth control signals generated according to a walking pattern of a pedestrian.

In addition, a pedestrian may have a walking rhythm which has a constant walking speed and stride, but in some cases, the pedestrian may have a walking rhythm which has an irregular walking speed and stride.

When a walking rhythm during walking of a pedestrian is the regular walking rhythm, the control unit 201 of the electromagnetic field vibration device 200 may normally generate the third control signal and the fourth control signal according to a walking speed and a stride during walking of the pedestrian.

However, when a walking rhythm during walking of a pedestrian is the irregular walking rhythm, the plurality of coils 202 driven by the third control signal and the fourth control signal may not effectively reduce fatigue of a sole of a foot of the pedestrian. Therefore, a driving method of the plurality of coils 202 for such cases will be described in detail below.

When a walking rhythm during walking of a pedestrian is the irregular walking rhythm, the control unit 201 of the electromagnetic field vibration device 200 may block the third control signal and the fourth control signal, may calculate each of a speed range and a stride range based on the maximum and minimum values of a walking speed of the pedestrian and the maximum and minimum values of a stride of the pedestrian, may calculate a current pulse period corresponding to the maximum value of a calculated speed range and a current pulse period corresponding to the minimum value of the calculated speed range based on a process of generating the third control signal and may generate a first alternating signal for causing currents with a pair of calculated current pulse periods to be alternately applied to the coil 202 provided on the sole 102 at a set interval. In addition, the control unit 201 of the electromagnetic field vibration device 200 may calculate an intensity value of a current corresponding to the maximum value of a calculated stride range and an intensity value of a current corresponding to the minimum value of the calculated stride range based on a process of generating the fourth control signal and may generate a second alternating signal for causing currents with a pair of calculated intensity values to be alternately applied to the coil 202 provided on the sole 102 at a set interval. As an example of the present invention, the set interval may be 1 second.

In this case, the plurality of coils 202 of the electromagnetic field vibration device 200 may be operated by overlapping and reflecting the first control signal, the second control signal, the first alternating signal, and the second alternating signal. Since the first control signal, the second control signal, the first alternating signal, and the second alternating signal are driven independently, the plurality of coils 202 may be driven by reflecting all the first control signal, the second control signal, the first alternating signal, and the second alternating signal which are generated according to a walking pattern of a pedestrian.

In this way, in the case of a pedestrian with an irregular walking rhythm, a pulse period and an intensity value of a current flowing in the plurality of coils 202 are adjusted every set interval in consideration of an irregular walking speed and stride range of a pedestrian to thus adjust the output interval and intensity of a pulsed electromagnetic field generated at the plurality of coils 202 for each set interval, thereby more efficiently reducing fatigue of a foot that is aggravated by the irregular walking of the pedestrian.

The shoe 1000 including the electromagnetic field vibration device according to one embodiment of the present invention may further include a plurality of reinforcing bands SB. The reinforcing band SB may include an elastic material, may be connected to the control unit 201, may extend from the sole 102, and may include the coil 202 at an end portion thereof. The reinforcing band SB may include an outer shell including an elastic material and a cable disposed in the outer shell and connected to the control unit 201, and the coil 202 may be provided at an end portion of the cable.

A plurality of connectors CN connected to the control unit 201 through the cable CA and a plurality of dummy connectors DM, which have the same shape as the connectors and are not connected to the control unit 201, may be provided at portions of an outer surface of the shoe body 101 adjacent to the sole 102. More specifically, the connector CN and the dummy connector DM may be provided adjacent to each other to form a pair of connector groups CNG in each of outer and inner surfaces of each of a front side portion and a rear side portion of the shoe body 101, and the connector CN and the dummy connector DM may be provided adjacent to each other to form a pair of connector groups CNG on a rear surface of the shoe body 101.

A band hole through which the reinforcing band SB passes may be formed in a portion of the sole 102 adjacent to each of a plurality of connector groups CNG. The above-described reinforcing band SB may pass through the band hole to protrude and extend to the outside, and a pedestrian may connect the end portion of the protruding reinforcing band SB to one of the connector CN and the dummy connector DM of the adjacent connector group CNG. In this case, when the pedestrian connects the end portion of the reinforcing band SB to the adjacent connector CN, since the coil 202 of the end portion of the reinforcing band SB is electrically connected to the control unit 201, a pulsed electromagnetic field may be formed around the coil 202, and when the pedestrian connects the end portion of the reinforcing band SB to the adjacent dummy connector DM, since the coil 202 of the end portion of the reinforcing band SB is electrically blocked from the control unit 201, a pulsed electromagnetic field may not be formed around the coil 202.

In this way, since the shoe of the present invention further includes the reinforcing band SB, a pulsed electromagnetic field generated at the coil 202 may be further supplemented in response to the above-described walking pattern of the pedestrian, thereby accelerating the recovery of foot fatigue due to the walking of the pedestrian. Hereinafter, the operation of the reinforcing band SB will be described in more detail.

As a foot landing method of a pedestrian is closer to a front end landing method, the control unit 201 of the electromagnetic field vibration device 200 may transmit a first recommendation signal to the pedestrian terminal 300, wherein the first recommendation signal is for recommending that the adjacent reinforcing band SB is connected to the connector CN included in the connector group CNG provided at the front side portion of the shoe body 101, and the remaining reinforcing band SB is connected to the dummy connector DM in the adjacent connector group CNG, and as a foot landing method of a pedestrian is closer to a rear end landing method, the control unit 201 of the electromagnetic field vibration device 200 may transmit the first recommendation signal to the pedestrian terminal 300, wherein the first recommendation signal is for recommending that the adjacent reinforcing band SB is connected to the connector CN included in the connector group CNG provided at each of the rear side portion and the rear surface of the shoe body 101, and the remaining reinforcing band SB is connected to the dummy connector DM in the adjacent connector group CNG.

When a foot landing method of a pedestrian is close to a front end landing method, it is difficult to completely recover the fatigue of the pedestrian with an electromagnetic field generated by the existing coils 202 excluding the coil 202 of the reinforcing band SB. Therefore, the control unit 201 of the present invention may generate the first recommendation signal for recommending that, to reinforce the electromagnetic field, the end portion of the adjacent reinforcing band SB is connected to the connector CN included in the pair of connector groups CNG provided at the front side portion, and the remaining reinforcing band SB is connected to the dummy connector DM in the adjacent connector group CNG, and the pedestrian may check the first recommendation signal through the pedestrian terminal 300 to connect the reinforcing band SB to the connector group CNG as recommended by the first recommendation signal.

On the other hand, when a foot landing method of a pedestrian is close to rear end landing, it is difficult to completely recover the fatigue of the pedestrian with an electromagnetic field generated by the existing coils 202 excluding the coils 202 of the reinforcing band SB. Therefore, the control unit 201 of the present invention may generate the first recommendation signal for recommending that, to reinforce the electromagnetic field, the end portion of the adjacent reinforcing band SB is connected to the connector CN included in the pair of connector groups CNG provided at the rear side portion and the rear surface, and the remaining reinforcing band SB is connected to the dummy connector DM in the adjacent connector group CNG, and the pedestrian may check the first recommendation signal through the pedestrian terminal 300 to connect the reinforcing band SB to the connector group CNG as recommended by the first recommendation signal.

As a pressure distribution during walking of a pedestrian is closer to an inner dense distribution, the control unit 201 of the electromagnetic field vibration device 200 may transmit a second recommendation signal to the pedestrian terminal 300, wherein the second recommendation signal is for recommending that the adjacent reinforcing band SB is connected to the connector CN included in the connector group CNG provided on the inner surface of each of the front side portion and the rear side portion of the shoe body 101, and the remaining reinforcing band SB is connected to the dummy connector DM in the adjacent connector group CNG. As a pressure distribution during walking of a pedestrian is closer to an outer dense distribution, the control unit 201 of the electromagnetic field vibration device 200 may transmit the second recommendation signal to the pedestrian terminal 300, wherein the second recommendation signal is for recommending that the adjacent reinforcing band SB is connected to the connector CN included in the connector group CNG provided on the outer surface of each of the front side portion and the rear side portion of the shoe body 101, and the remaining reinforcing band SB is connected to the dummy connector DM in the adjacent connector group CNG.

When a pressure distribution during walking of a pedestrian is close to an inner dense distribution, it is difficult to completely recover fatigue of the pedestrian with an electromagnetic field generated by the existing coils 202 excluding the coil 202 of the reinforcing band SB. Therefore, the control unit 201 of the present invention may generate the second recommendation signal for recommending that, to reinforce the electromagnetic field, the end portion of the adjacent reinforcing band SB is connected to the connector CN included in the pair of connector groups CNG provided on the inner surface of the front side portion and the rear side portion, and the remaining reinforcing band SB is connected to the dummy connector DM in the adjacent connector group CNG, and the pedestrian may check the second recommendation signal through the pedestrian terminal 300 to connect the reinforcing band SB to the connector group CNG as recommended by the second recommendation signal.

On the other hand, when a pressure distribution during walking of a pedestrian is close to an outer dense distribution, it is difficult to completely recover fatigue of the pedestrian with an electromagnetic field generated by the existing coils 202 excluding the coil 202 of the reinforcing band SB. Therefore, the control unit 201 of the present invention may generate the second recommendation signal for recommending that, to reinforce the electromagnetic field, the end portion of the adjacent reinforcing band SB is connected to the connector CN included in the pair of connector groups CNG provided on the outer surface of the front side portion and the rear side portion, and the remaining reinforcing band SB is connected to the dummy connector DM in the adjacent connector group CNG, and the pedestrian may check the second recommendation signal through the pedestrian terminal 300 to connect the reinforcing band SB to the connector group CNG as recommended by the second recommendation signal.

The reason why the pedestrian connects the reinforcing band SB to the dummy connector DM in response to the first and second recommendation signals is that, since the pedestrian may experience discomfort due to the reinforcing band SB when walking because the reinforcing band SB protrudes to the outside as described above, such discomfort can be prevented by connecting and fixing the reinforcing band SB to the dummy connector DM.

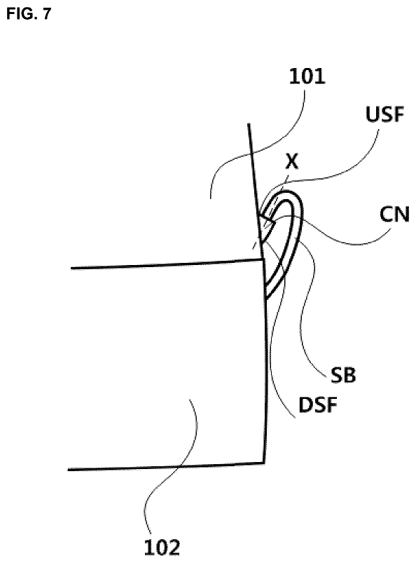

FIG. 7 is an enlarged view illustrating a state in which the reinforcing band SB is connected to the connector CN according to one embodiment of the present invention.

The connector CN provided in the shoe of the present invention may have an upper surface USF that protrudes downward from the outer surface of the shoe body 101 to form a predetermined acute angle with the outer surface of the shoe body 101 and has a hole into which the end portion of the reinforcing band SB is inserted, and a lower surface DSF that connects an end portion of the upper surface USF and the outer surface of the shoe body 101. When the end portion of the reinforcing band SB is inserted into the hole formed in the upper surface USF of the connector CN, the coil 202 provided at the end portion of the reinforcing band SB may be fastened to the connector CN and electrically connected to the control unit 201. When the end portion of the reinforcing band SB is fastened to the connector CN, a direction of a virtual central axis X of an electromagnetic field generated at the coil 202 provided at the end portion of the reinforcing band SB may be a direction perpendicular to the upper surface USF of the connector CN and thus may be directed to an inner bottom surface of the shoe body 101.

In this way, since the upper surface USF of the connector CN in the present invention forms a predetermined acute angle with the outer surface of the shoe body 101, when the end portion of the reinforcing band SB is fastened to the connector CN, the virtual central axis X of a pulsed electromagnetic field generated from the end portion of the reinforcing band SB is formed in a direction perpendicular to the upper surface USF, and thus the pulsed electromagnetic field generated from the end portion of the reinforcing band SB is output toward a foot of a pedestrian, thereby more efficiently reducing fatigue of a foot caused by the walking of the pedestrian.

According to the present invention, a plurality of coils provided according to the present invention are differently driven in response to a calculated walking pattern of a pedestrian to generate a pulsed electromagnetic field, thereby effectively reducing fatigue caused by walking of the pedestrian.

Furthermore, in the present invention, in the case of a pedestrian with an irregular walking rhythm, a pulse period and an intensity value of a current flowing in a plurality of coils are adjusted every set interval in consideration of an irregular walking speed and stride range of a pedestrian to thus adjust the output interval and intensity of a pulsed electromagnetic field generated at the plurality of coils every set interval, thereby more efficiently reducing fatigue of a foot that is aggravated by the irregular walking of the pedestrian.

The above-described embodiments are for illustrative purposes, and it will be understood by those skilled in the art that the embodiments can be easily modified into other specific forms without changing the technical idea or essential features of the above-described embodiments. Accordingly, it should be understood that the above-described embodiments are exemplary in all respects and not restrictive. For example, each component in a single type can be implemented in a distributed manner. Likewise, components in a distributed type can be implemented in a combined manner.

The scope sought to be protected through the present specification is defined not by the detailed description but by the appended claims, and all modifications or alterations derived from the concept, the range, and the equivalents of the claims will be construed as being included in the scope of the present invention.

What is claimed is:

1. A shoe from which a pulsed electromagnetic field is generated and which is a shoe using a pulsed electromagnetic field technology for generating vibrations using a pulsed electromagnetic field, the shoe comprising:
a shoe body;
a sole provided below the shoe body;
a battery module provided on the sole and configured to supply DC power;
an electromagnetic field vibration device provided on the sole and connected to the battery module through a cable; and
a sensing module provided on the sole and connected to the battery module through a cable,
wherein, while a user wearing the shoe walks, the sensing module calculates a walking pattern of a pedestrian based on sensing data transmitted from the shoe,
the electromagnetic field vibration device includes a plurality of coils and a control unit configured to control the plurality of coils,
the control unit of the electromagnetic field vibration device is disposed to overlap a narrowest portion of a middle portion of the sole, the plurality of coils of the electromagnetic field vibration device are disposed to be distributed at a front end portion of the sole and a rear end portion of the sole and are connected to the control unit of the electromagnetic field vibration device through a cable, and
the control unit of the electromagnetic field vibration device generates a pulsed electromagnetic field in different manners through the plurality of coils according to the calculated walking pattern.

2. The shoe of claim 1, further comprising a switch module connected to the battery module through a cable to control the electromagnetic field vibration device to be turned on or off,
wherein the control unit is connected to a pedestrian terminal used by the pedestrian through a network by using a provided communication module and transmits operation information indicating an operation state of the plurality of coils to the pedestrian terminal in real time,
the shoe body includes a fixture for attachment and separation of the switch module,
when a time for which the user wearing the shoe walks satisfies a set time, the sensing module calculates the walking pattern of the pedestrian based on the sensing data during the time,
the walking pattern includes information about a foot landing method, a pressure distribution during walking, a walking rhythm, a walking speed, and a stride, and
the number of the coils disposed at the front end portion of the sole is greater than the number of the coils disposed at the rear end portion of the sole.

3. The shoe of claim 2, wherein the foot landing method of the walking pattern is classified into a front end landing method, a middle landing method, and a rear end landing method,
the pressure distribution during walking of the walking pattern is classified into a uniform distribution, an inner dense distribution, and an outer dense distribution,
the walking rhythm of the walking pattern is classified into a regular walking rhythm which has a constant walking speed and stride and irregular walking rhythm which has an irregular walking speed and stride,
the walking speed of the walking pattern is classified into a high speed, an average speed, and a low speed, and
the stride of the walking pattern is classified into a long stride, an average stride, and a short stride.

4. The shoe of claim 3, wherein, as the foot landing method of the pedestrian is closer to the front end landing method, the control unit of the electromagnetic field vibration device generates a first control signal for causing a higher current to flow in the coils disposed at the front end portion of the sole as compared to other coils,
as the pressure distribution during walking of the pedestrian is closer to the inner dense distribution, the control unit of the electromagnetic field vibration device generates a second control signal for performing control such that a higher current flows in the coils disposed at an inner side of the sole as compared to other coils,
as the walking speed during walking of the pedestrian is closer to the high walking speed, the control unit of the electromagnetic field vibration device generates a third control signal for performing control such that a pulse period of a current flowing in the coil provided on the sole becomes shorter,
as the stride during walking of the pedestrian becomes longer, the control unit of the electromagnetic field vibration device generates a fourth control signal for performing control such that intensity of a current flowing in the coil provided on the sole is increased, and
the plurality of coils of the electromagnetic field vibration device are operated by overlapping and reflecting the first to fourth control signals.

5. The shoe of claim 4, wherein, when the walking rhythm during walking of the pedestrian is the regular walking rhythm, the control unit of the electromagnetic field vibration device normally generates the third control signal and the fourth control signal according to a walking speed and a stride during walking of the pedestrian,
when the walking rhythm during walking of the pedestrian is the irregular walking rhythm, the control unit of the electromagnetic field vibration device blocks the third control signal and the fourth control signal and calculates each of a speed range and a stride range based on a maximum value and a minimum value of the walking speed of the pedestrian and a maximum value and a minimum value of the stride of the pedestrian,
when the walking rhythm during walking of the pedestrian is the irregular walking rhythm, the control unit of the electromagnetic field vibration device calculates a current pulse period corresponding to a maximum value of a calculated speed range and a current pulse period corresponding to a minimum value of the calculated speed range based on a process of generating the third control signal and generates a first alternating signal for causing currents with a pair of current pulse periods to be alternately applied to the coil provided on the sole at a set interval,
the control unit of the electromagnetic field vibration device calculates an intensity value of a current corresponding to a maximum value of a calculated stride range and an intensity value of a current corresponding to a minimum value of the calculated stride range based on a process of generating the fourth control signal and generates a second alternating signal for causing currents with a pair of intensity values to be alternately applied to the coil provided on the sole at a set interval,
the plurality of coils of the electromagnetic field vibration device are operated by overlapping and reflecting the first control signal, the second control signal, the first alternating signal, and the second alternating signal, and
the set interval is an interval of 1 second or less.

6. The shoe of claim 5, further comprising a plurality of reinforcing bands which include an elastic material, are connected to the control unit, extend from the sole, and include coils at end portions thereof,
wherein a plurality of connectors connected to the control unit through a cable and a plurality of dummy connectors which have the same shape as the connectors and are not connected to the control unit are provided at portions of an outer surface of the shoe body adjacent to the sole,
the connector and the dummy connector are provided adjacent to each other to form a pair of connector groups on each of an outer surface and an inner surface of each of a front side portion and a rear side portion of the shoe body,
the connector and the dummy connector are provided adjacent to each other to form a pair of connector groups on a rear surface of the shoe body,
a band hole through which the reinforcing band passes is formed in a portion of the sole adjacent to each of a plurality of connector groups,
the pedestrian connects each reinforcing band, which passes through the band hole to protrude and extend to the outside, to any one of the connector and the dummy connector of an adjacent connector group,
when the pedestrian connects the end portion of the reinforcing band to an adjacent connector, the coil of the end portion of the reinforcing band is electrically connected to the control unit so that a pulsed electromagnetic field is formed around the coil, and
when the pedestrian connects the end portion of the reinforcing band to an adjacent dummy connector, the coil of the end portion of the reinforcing band is electrically blocked from the control unit.

7. The shoe of claim 6, wherein as the foot landing method of the pedestrian is closer to the front end landing method, the control unit of the electromagnetic field vibration device transmits a first recommendation signal to the pedestrian terminal, wherein the first recommendation signal is for recommending that an adjacent reinforcing band is connected to the connector included in the connector group provided at the front side portion of the shoe body, and the remaining reinforcing band is connected to the dummy connector in an adjacent connector group,
as the foot landing method of the pedestrian is closer to the rear end landing method, the control unit of the electromagnetic field vibration device transmits the first recommendation signal to the pedestrian terminal, wherein the first recommendation signal is for recommending that the adjacent reinforcing band is connected to the connector included in the connector group provided at each of the rear side portion and the rear surface of the shoe body, and the remaining reinforcing band is connected to the dummy connector in the adjacent connector group,
as the pressure distribution during walking of the pedestrian is closer to the inner dense distribution, the control unit of the electromagnetic field vibration device transmits a second recommendation signal to the pedestrian terminal, wherein the second recommendation signal is for recommending that the adjacent reinforcing band is connected to the connector included in the connector group provided on the inner surface of each of the front side portion and the rear side portion of the shoe body, and the remaining reinforcing band is connected to the dummy connector in the adjacent connector group,
as the pressure distribution during walking of the pedestrian is closer to the outer dense distribution, the control unit of the electromagnetic field vibration device transmits the second recommendation signal to the pedestrian terminal, wherein the second recommendation signal is for recommending that the adjacent reinforcing band is connected to the connector included in the connector group provided on the outer surface of each of the front side portion and the rear side portion of the shoe body, and the remaining reinforcing band is connected to the dummy connector in the adjacent connector group,
the connector has an upper surface that protrudes downward from the outer surface of the shoe body to form a predetermined acute angle with the outer surface of the shoe body and has a hole into which the end portion of the reinforcing band is inserted, and a lower surface configured to connect an end portion of the upper surface and the outer surface of the shoe body,
when the end portion of the reinforcing band is inserted into the hole formed in the upper surface of the connector, the coil provided at the end portion of the reinforcing band is fastened to the connector and electrically connected to the control unit, and
when the end portion of the reinforcing band is fastened to the connector, a direction of a virtual central axis of an electromagnetic field generated at the coil provided at the end portion of the reinforcing band is a direction perpendicular to the upper surface of the connector to be directed to an inner bottom surface of the shoe body.
